(19) United States
(12) Patent Application Publication
(10) Pub. No.: US 2025/0095778 A1
(43) Pub. Date: Mar. 20, 2025

(54) PLATFORM FOR ANALYSIS OF HIGH-THROUGHPUT SEQUENCING DATA

(71) Applicants: Merck Sharp & Dohme LLC, Rahway, NJ (US); MSD Czech Republic s.r.o., Prague (CZ); MSD Biotech B.V., Oss (NL)

(72) Inventors: Paul Alan Duncan, Mount Joy, PA (US); Julia Meredith Maritz, Boston, MA (US); Geoffrey Daniel Hannigan, Wilmington, MA (US); Christopher Harron Woelk, Winchester, MA (US); Christopher James Wang, Norristown, PA (US); Jack Benjamin Baker, Phoenixville, PA (US); Vanessa Vazquez Sarathy, Wyndmoor, PA (US); Ron Šmeral, Prague (CZ); Ales Vondra, Prague (CZ); Ondrej Klempir, Uvaly (CZ); Ondrej Tupa, Prague (CZ); Anna Gromek, Prague (CZ); Danny Asher Bitton, Prague (CZ); Jakob Moritz Goldmann, Nijmegen (NL)

(21) Appl. No.: 18/886,133

(22) Filed: Sep. 16, 2024

Related U.S. Application Data
(60) Provisional application No. 63/583,004, filed on Sep. 15, 2023.

(51) Int. Cl. G16B 30/00

(52) U.S. Cl. CPC G16B 30/00

(57) ABSTRACT

A sequencing data analysis platform can process datasets that include a large number of sequence read. The reads are aligned to one or more reference genomes. Due to sequencing errors, sequencing noise, or genuine differences between a reference genome and the individual species being sequenced, this mapping process may tolerate a certain number of mismatches, insertions, or deletions. The sequencing data analysis platform provides a set of tools for analyzing and visualizing the sequence reads.

### CROSS-REFERENCE TO RELATED APPLICATIONS

This application claims the benefit of Provisional Patent Application No. 63/583,004, filed Sep. 15, 2023, which is incorporated by reference.

### BACKGROUND

### 1. Technical Field

The subject matter described relates generally to genetic sequencing and, in particular, to a platform for analysis of high-throughput sequencing data.

### 2. Background Information

There are a wide range of use cases where detection of specific genetic sequences is of value. One significant use case is the detection of genetic sequences that indicate the presence of a particular virus in a sample. Existing approaches to detection of specific genetic sequences suffer from a range of limitations. For example, in vitro screening for viruses is targeted to a single virus and is both time consuming and costly to perform. Various forms of microscopy (e.g., transmission electron microscopy) may be used but such approaches have low throughput and can be unreliable with regards to consistent detection. Another common approach, polymerase chain reaction (PCR) testing, is also limited to specific and limited ranges. Next generation sequencing (NGS) can produce high-throughput sequencing data, but existing analysis techniques are insufficient to take advantage of the throughput capabilities of these approaches. There is thus a need for high-throughput analysis that can pair with NGS to unlock the full potential of NGS techniques.

### SUMMARY

The above and other problems may be addressed by a sequencing data analysis platform. Sequencing datasets typically include large numbers of short DNA sequences, or “reads.” These reads are mapped (aligned) to a reference genome or to a database of reference genomes. Due to sequencing errors, sequencing noise, or genuine differences between a reference genome and the individual species being sequenced, this mapping process may tolerate a certain number of mismatches, insertions, or deletions. Thus, the analysis performed by the platform enables matches to be found between reads and the reference genome where there is less than 100% identity between the reads and corresponding portions of the reference genome.

In various embodiments, the platform provides tools including a short read alignment (SRA) tool that allows users to align the reads from a vaccine or biologics-associated sample (e.g., cell bank lots) to viral reference databases. This allows detection of potential viral contamination in the samples. The platform may also provide one or more of protein alignment analysis, de novo assembly, or statistical analysis of single nucleotide variation (SNV) within a sample relative to the reference genome. The platform may also provide users with multiple result formats including viewable tables and an interactive viewer.

In one embodiment, the platform includes a system with one or more processors, an interface module, and one or more tools. The interface module interacts with the one or more processors to obtain biomolecule sequencing data including sequence reads. For example, the interface module may obtain the sequence reads from a sequencing system such as a next generation sequencer. The one or more tools interact with the one or more processors to process the biomolecule sequencing data by performing analysis on the sequencing data, including dividing the sequence reads into subsets. The interface module provides a web-based interface that causes a client device to display results of analysis, including data derived from the subsets of the sequence reads.

The tools can include a short read alignment module that classifies the sequence reads into the subsets based on alignment to one or more reference genomes. The short read alignment module receives a readset including the sequence reads, preprocesses the readset, attempts alignment of the sequence reads with one or more reference genomes, and classifies the sequence reads into the subsets based on results of the attempts to align the plurality of reads. The subsets can include aligned, half-aligned, and unaligned sequence reads relative to the one or more reference genomes. Aligned reads are ones that generate complete matches with the one or more reference genomes, half-aligned reads are ones for which only one part are aligned with the one or more reference genomes, and unaligned reads are any other reads.

In some embodiments, the short read alignment module provides a multiple aligners for alignment of the sequence reads with the one or more reference genomes. The user interface may provide controls for a user to select one or more of the aligners for use by the short read alignment module in analysis of the sequence reads. The short read alignment module may calculate alignment statistics for one or more reference organisms represented in the one or more reference genomes with which at least some of the sequence reads were aligned, enrich the alignment statistics using a taxonomy for the one or more reference organisms, generate one or more coverage plots for the sequence reads relative to the one or more reference organisms, and build one or more consensus contigs from the sequence reads for the one or more reference organisms. The alignment statistics may be stored in a file and enriched by adding one or more of: the taxonomy, a total number of reads per family, a total number of reads per genus, or a total number of reads per species.

The tools may also include a protein alignment module that matches sequences of amino acids in the sequencing data to reference proteins, a de novo assembly module that builds a genome from the sequence data, and a single nucleotide variation module that identifies variations in the sequence reads relative to the one or more reference genomes and generates a visualization representing the variations.

In one embodiment, the de novo assembly module builds the genome from the sequence data by performing a series of operations to receive the sequence reads, produce aligned contigs relative to the one or more genomes from the sequence reads, generate coverage plots indicating coverage of the one or more reference genomes in the sequence reads, and provide a taxonomy table generated from the aligned contigs and the coverage plots. The taxonomy table identifies organisms identified in the sequence reads. The taxonomy table may also include clickable links to the underlying sequencing data for each organism.

The visualization generated by the single nucleotide variation module may include a variations plot that indicates, for each position in a genome of the one or more genomes, metrics describing an amount of coverage for that position in the sequence reads and a degree of variation between the sequence reads and the genome. Additionally or alternatively, the visualization may include a table with one or more of: a nucleotide position, a reference allele, a variant allele, a variant type, a frequency, nucleotide context, an amino acid position, a coding sequence, a strandedness of an affected amino acid, a reference amino acid, a variant amino acid, an indication of whether the change is synonymous, an amino acid change, a protein context, an amino acid property change, or a change effect. To generate the visualization, the single nucleotide variation module may receive the sequence reads, obtain parameters for analysis of the sequence reads, calculate variation metrics with a comparison of the plurality of reads to the one or more reference genomes, and produce the visualization using the variation metrics.

In some embodiments, the system also includes a curation module. A user can use a user interface provided by the curation module to identify portions of a reference genome that should be ignored or given a lesser weighting in analysis because those portions correspond to contamination, noise, or a low-complexity region (meaning one that appears in many genomes substantially unchanged).

### BRIEF DESCRIPTION OF THE DRAWINGS

FIG. 1 is a block diagram of a networked computing environment suitable for providing a high-throughput sequencing data analysis platform, according to one embodiment.

FIG. 2 is a block diagram of the analysis system of FIG. 1, according to one embodiment.

FIG. 3 is a flowchart of a method for performing short read alignment, according to one embodiment.

FIG. 4 is a flowchart of a method for performing de novo assembly, according to one embodiment.

FIG. 5 is a flowchart of a method for analysis single nucleotide variations in a sample, according to one embodiment.

FIG. 6 is a block diagram illustrating an example of a computer suitable for use in the networked computing environment of FIG. 1, according to one embodiment.

### DETAILED DESCRIPTION

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### Overview

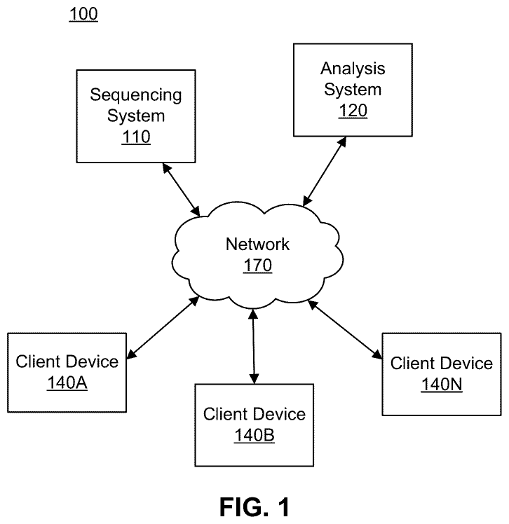

FIG. 1 illustrates one embodiment of a networked computing environment 100 suitable for providing a high-throughput sequencing data analysis platform. In the embodiment shown, the networked computing environment 100 includes a sequencing system 110, an analysis system 120, and one or more client devices 140, all connected via a network 170. FIG. 1 shows three client device 140 (a first client device 140A, a second client device 140B, and an Nth client device 140N), but the networked computing environment 100 can include any number of such devices. In other embodiments, the networked computing environment 100 includes different or additional elements. In addition, the functions may be distributed among the elements in a different manner than described.

The sequencing system 110 is one or more devices that sequence biopolymers (e.g., DNA or RNA) to produce sequence data. In one embodiment, the sequencing system 110 includes a NGS machine. Biopolymers are extracted from a sample and cut into fragments. The NGS machine sequences each fragment to generate a readset including a large number of sequence reads, each sequence read representing the sequence of nucleotides of one of the fragments. As described previously, the sequence reads may contain sequencing errors where the nucleotide indicated by the sequence read for a particular position within a fragment does not match the actual nucleotide at that position in the fragment. A person having ordinary skill in the art would appreciate that a range of sequencing techniques and technologies may be used to generate sequence reads from biopolymer fragments.

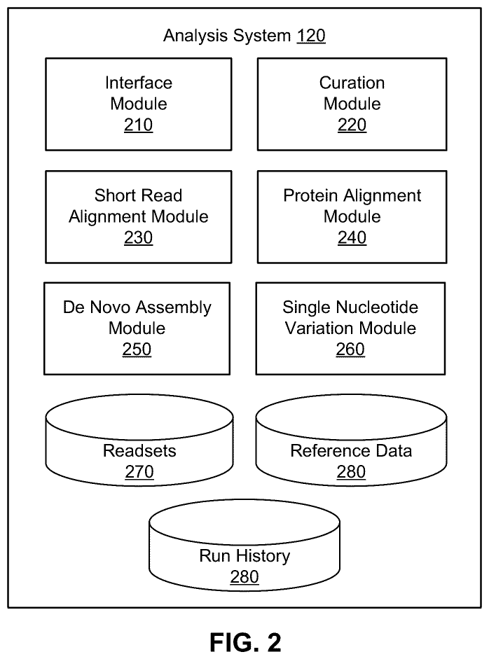

The analysis system 120 includes one or more computing devices that analyze readsets generated by the sequencing system 110. In one embodiment, the analysis system 120 provides a web-based application for analysis of high-throughput sequencing data, such as data from viral metagenomic studies. The web-based application may include a user interface that is accessed via the network 170 using a client device 140. The user interface may include a tab-based interface that provides access to results from several different analysis tools. Using the tab-based interface, authorized users may select one or more readsets for analysis, set analysis parameters for tools, and review the results of the analysis generated by the tools, etc. The application may be used to support GxP and non-GxP analysis and detection of viral adventitious agents using genomic data. For example, the platform may be used to test vaccine samples using High Throughput Sequencing (HTS) instruments to screen for viral contaminants. Various embodiments of the analysis system 120 are described in greater detail below, with reference to FIG. 2.

A client device 140 is a computing device with which a user interacts with the user interface provided by the analysis system 120. In one embodiment, the client device 140 presents the user interface in a web-browser. Alternatively, the user interface may be provided by dedicated software executing on the client device 140. In either case, the browser or other software may interact with the analysis system 120 using an application programming interface (API). The user may identify readsets for analysis and the corresponding parameters to use and view the results of previously performed analyses in the user interface.

In some embodiments, the client device 140 also provides a user interface with which the user can transfer readsets from the sequencing system 110 to the analysis system 120 (e.g., by copying the readsets to a shared network drive accessible by both the sequencing system 110 and the analysis system 120 or copying them directly to a local drive of the analysis system 120, depending on the specific permissions and configuration of the networked computing environment 100).

The network 170 provides the communication channels via which the other elements of the networked computing environment 100 communicate. The network 170 can include any combination of local area and wide area networks, using wired or wireless communication systems. In one embodiment, the network 170 uses standard communications technologies and protocols. For example, the network 170 can include communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 170 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 170 may be represented using any suitable format, such as hypertext markup language (HTML) or extensible markup language (XML). In some embodiments, some or all of the communication links of the network 170 may be encrypted using any suitable technique or techniques.

### Example Analysis System

FIG. 2 illustrates one embodiment of the analysis system 120. In the embodiment shown, the analysis system 120 includes an interface module 210, a curation module 220, a short read alignment (SRA) module 230, a protein alignment module 240, a de novo assembly module 250, a single nucleotide variation module 260, and datastores for readsets 270, reference data 280, and run history 290. In other embodiments, the analysis system 120 includes different or additional elements. In addition, the functions may be distributed among the elements in a different manner than described.

The interface module 210 drives the user interface with which users access the functionality provided by the other modules of the analysis system 120. The interface module 210 may provide an API with which software (e.g., a browser) executing on the client devices interacts with the analysis system 120. The interface module 210 may provide credential verification (e.g., requiring a username and password) to ensure only authorized users access the analysis system 120. The interface module 210 may also provide controls to enable users to import sequence read data (e.g., from the sequencing system 110). In one embodiment, the interface module 210 provides a web-based user interface with tabs that enable a user to access some or all of functionality provided by the other modules.

The curation module 220 enables users to curate the reference data 280 used in analysis. Reference data 280 (e.g., genomes) are often contaminated with DNA from other sources, such as culture hosts and vectors from sequencing processes or reagents. Furthermore, many genomes share similar regions, referred to as “low-complexity regions.” This can lead to false positives. To address this, in one embodiment, the curation module 220 provides controls to enable a user to remove data corresponding to contamination from reference data, identify low-complexity regions, or both. For example, portions of the reference data 280 corresponding to contamination may be identified by a user and removed or tagged with metadata indicating they are likely due to contamination. Similarly, portions of the reference data 280 identified as corresponding to low-complexity regions may be identified by the user or selected automatically based on one or more databases of known low-complexity regions and tagged with metadata indicating they are low-complexity regions. Portions of the reference data 280 that are tagged as contamination or corresponding to low-complexity regions can either be ignored or given less weight than other portions of the reference data 280 during analysis by the other modules. Alternatively, matching of low-complexity regions between a sample and reference data 280 may be required but not sufficient for a positive match to be found.

The SRA module 230 attempts to align each read in a readset with one or more reference genomes (e.g., in the reference data 280) and classifies the reads into subsets based on the results of the alignment. In one embodiment, each read is classified as aligned, half-aligned, or unaligned with a reference genome. Aligned reads are ones that generate matches between readset sequences and reference organism sequences. Half-aligned reads are ones for which only one part of the readset sequences (one-mate) are aligned with reference organism sequences. Other reads are unaligned reads, such as reads due to sequencing errors, randomness, or noise as well as reads that align with organisms (e.g., viruses) with no organism ID found in the reference data 280. The SRA module 230 may also use a taxonomy and coverage plots to make consensus contigs that are the best match for the whole readset. The alignment results generated by the SRA module 230 may be formatted and presented in a tab in the user interface provided by the interface module 210.

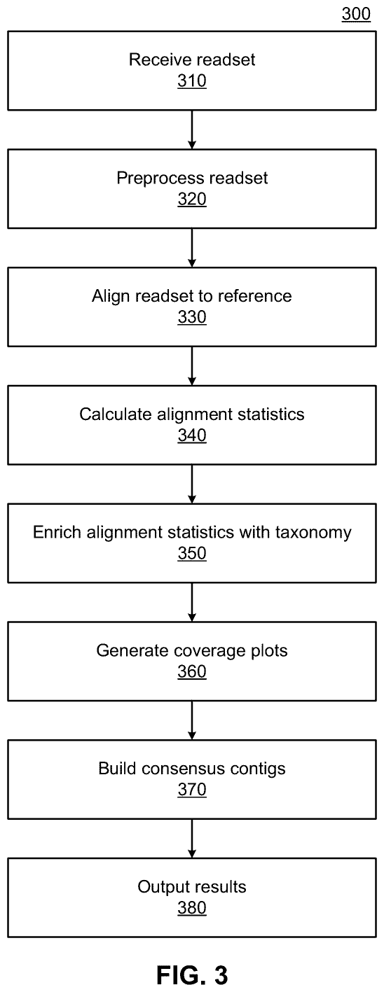

FIG. 3 illustrates an example method 300 that may be performed by the SRA module 230, according to one embodiment. Although the steps of FIG. 3 are described from the perspective of the SRA module 230 performing the method 300, some or all of the steps may be performed by other entities or components. In addition, some embodiments may perform steps in parallel, perform steps in different orders, or perform different steps.

In the embodiment shown in FIG. 3, the method 300 begins with the SRA module 230 receiving 310 one or more readsets. Depending on the configuration, the SRA module 230 may access the readsets from a remote storage location (e.g., a database associated with the sequencing system 210), access a copy of the readsets already saved to local storage (e.g., in readsets 270) by a user, or receive an indication of a remotely stored readset and make a local copy before proceeding with the analysis.

In some embodiments, the SRA module 230 preprocesses 320 the readsets. Preprocessing converts the readsets into a predetermined format to aid in efficient analysis. Preprocessing may include deduplicating read names to ensure each read has a unique identifier, removing suffixes from read names, and the like.

The SRA module 230 aligns 330 the reads to a reference database (e.g., in reference data 280). The SRA module 230 may use an alignment algorithm such as Bowtie or MagicBLAST. In some embodiments, the user can select between two or more possible alignment algorithms. The user may also provide parameters to configure the selected alignment algorithm. The SRA module 230 may also filter the aligned reads to remove reads that do not meet one or more criteria. In one embodiment, the filter criteria include one or more of whether both mates of a read pair are aligned, and aligned in “proper pair” (as defined by the specific aligner used), whether the alignment is primary or secondary, or the alignment score.

The SRA module 230 calculates 340 alignment statistics for each accession from the (potentially filtered) aligned reads. The alignment statistics may be stored in a Pileup file or any other suitable format. In one embodiment, alignments are aggregated by reference accession and the statistics include one or more of: the average GC ratio of reads mapped to this scaffold, the median fold coverage of this scaffold, the percent of the scaffold with any coverage, the number of bases with any coverage, the number of read mapped to a plus strand, the number of reads mapped to a minus strand, or the standard deviation of coverage. The generated statistics may be enriched 350 with additional data based on a taxonomy. The enriched data may include one or more of an enhanced version of the Pileup (or other type of) file including the taxonomy, a total number of reads per family, a total number of reads per genus, and a total number of reads per species. In this context, a taxonomy is a taxonomic classification of a given organism and enhancing means adding columns to the data for the taxonomic ranks of the organism. For example, for a pileup entry containing a column with the accession ID: AY699793.1, the data may be enhanced with columns indicating superkingdom=viruses, family=cicoviridae, genus=circovirus, and species=porcine circovirus 2. The taxonomy data may be obtained from public sources (e.g., NCBI) or provided by users.

The SRA module 230 generates 360 coverage plots indicating a number of reads that overlap with each position in the genome. In one embodiment, the SRA module 230 generates 360 a BAM file and a FASTA file for each accession to store the coverage plot data. The SRA module 230 may also build 370 consensus contigs from the reads in the readset. For example, in one embodiment, the SRA module 230 may build 370 a consensus contig by, for each aligned accession: creating a VCF file, then building a normalized BCF file, and from there generating a normalized BCF file without adjacent indels (e.g., within 5 bp), a consensus FASTA file of the whole genome, and a consensus FASTA file of only covered regions (as indicated by the coverage plots). Alternatively, as another example, the SRA module 230 may generate a consensus FASTA file (single record, the complete reference genome of an accession, with consensus sequences where there is variation) and a consensus contigs FASTA file (potentially multiple records, containing the individual consensus sequences for contiguous regions (contigs) covered by alignments).

The SRA module 230 may output 380 results based on the analysis. For example, the SRA module 230 may create files with the identified subsets of reads, such as all of the aligned and half-aligned reads for each of one or more genomes in the reference data 280. The output may also include a file indicating the number of hits for one or more organisms. In one embodiment, results may be displayed in a “pivot table” that is generated by taking the taxonomy-enriched pileup, and reshaping it. Once reshaped, the entries are aggregated by species (by superkingdom-family-genus-species key) and, for each species, the SRA module 230 calculates the accession with the most alignments, the accession with the highest coverage, and the total number of alignments (of all accessions of that species). Thus, the resulting shape of the table is “superkingdom,” “family,” “genus,” “species,” “Max_Coverage,” “Max_Coverage_Accession.” “Max_Hits,” “Max_Hits_Accession,” “Sum_Of_Hits.”

Referring back to FIG. 2, the protein alignment module 240 perform a similar function to the SRA module 230 but matches sequences of amino acids in NGS protein data to reference proteins rather than nucleotide sequences in DNA fragments to reference genomes. Protein sequences are more conserved than nucleotide sequences and thus can provide additional information about what is present in a sample with less sensitivity to mutations. In one embodiment, this pipeline generates output including: a Pileup file, a pivot table, and coverage plots. The Pileup file is similar to that generated by the SRA module 230 but may use variations of the algorithm used to provide optimized output shape for the use case. The pivot table may be broken down by organism (e.g., virus accession) and then further by proteins of that organism (protein accession). For each organism, the pivot table can include total reads aligned to it and the ratio of found proteins vs. total “known” (e.g, present in NCBI taxonomy proteins of that organism). For each protein, the pivot table can include the number of reads aligned to it and the coverage percentage. The coverage plots can be the same or similar to those generated by the SRA module 230.

The de novo assembly module 250 combines information from reads in a read set to assemble genomes from the reads of fragments. The de novo assembly module 250 may also build a taxonomy table. The taxonomy table can be built using coverage plots of each accession that are based on the contigs and reads in the sequencing data. The taxonomy table can identify the accessions corresponding to different elements of the taxonomy and provide access to the underlying sequencing data.

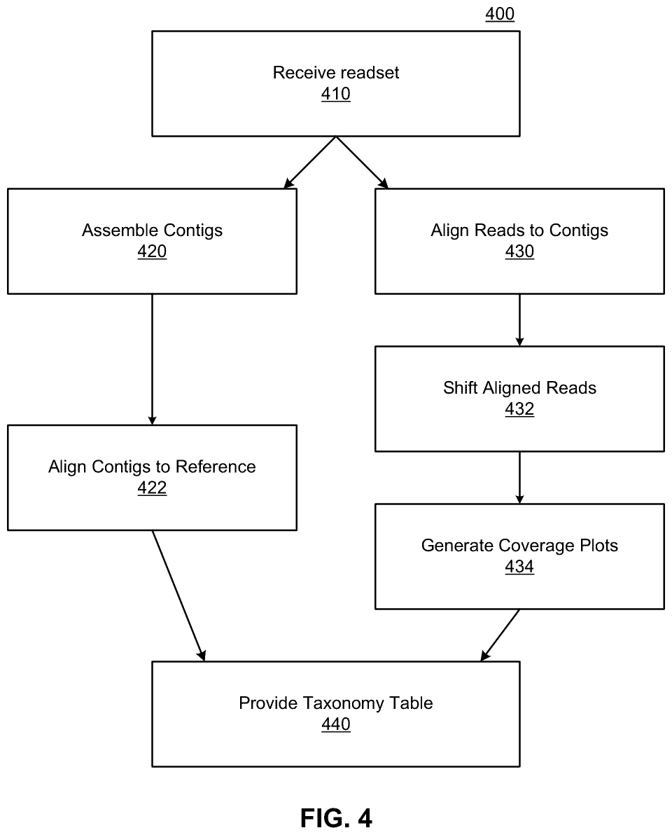

FIG. 4 illustrates an example method 400 that may be performed by the de novo assembly module 250, according to one embodiment. Although the steps of FIG. 4 are described from the perspective of the de novo assembly module 250 performing the method 400, some or all of the steps may be performed by other entities or components. In addition, some embodiments may perform steps in parallel, perform steps in different orders, or perform different steps.

In the embodiment shown, the method 400 begins with the de novo assembly module 250 receiving 410 one or more readsets. The readsets may be generated by the sequencing system 110 and selected by a user for analysis. As described previously, the readsets may be accessed remotely or a local copy may be stored by the analysis system 120 (e.g., in the readsets 270).

Regardless of exactly how the readsets are obtained, the de novo assembly module 250 performs two parallel analyses. Note that in this context, parallel just means that the two branches of the analysis are performed independently and the results later combined. The branches do not need to be performed simultaneously. Rather, one may be performed after the other, but parallel analysis may improve the overall efficiency of the method 400.

In one branch, the de novo assembly module 250 assembles 420 contigs from the readsets and aligns 422 the contigs to a reference genome. In one embodiment, the contigs are assembled 420 using Megahit and aligned to the Reference Viral Database (RVDB) using MagicBLAST. In the other branch, the de novo assembly module 250 aligns 430 reads to contigs (e.g., using Bowtie), shifts 432 the aligned reads, and generates 434 coverage plots for each accession. The coverage plots can indicate coverage as determined from both the contigs and the reads.

The de novo assembly module 250 uses the analysis performed in each branch to provide 440 the taxonomy table. The taxonomy table may identify accessions identified in the readset and include clickable links to the underlying sequencing data for each accession.

Referring once again to FIG. 2, the Single Nucleotide Variation (SNV) module 260 identifies variations in the reads of one or more readsets 270 relative to the reference data 280. As noted previously, sequencing errors, sequencing noise, and genuine differences between a reference genome and the individual species being sequenced can all lead to differences between the readsets 270 and the reference data 280. The SNV module 260 aids the user in identifying and evaluating these variations. In one embodiment, the SNV module 260 generates a variations plot that indicates, for each position in a genome, metrics describing the amount of coverage for that position in the readset and a degree of variation between the reads in the readset and the reference genome. This may also aid in the detection of mutations and other genetic changes in an organism's genome over time.

The SNV module 260 may additionally or alternatively produce a table of variation data. The table may include some or all of the following information:

| | |
|---|---|
| Position (nucleotide) | Position of the nucleotide in the sequence |
| Reference allele | Original allele in the reference |
| Variant allele | Changed allele in the sample |
| Variant type | Type of change: substitution, insertion, or deletion |
| Frequency (%) | Frequency of the variant |
| Nucleotide | Two 30-nucleotide long sequences with the top |
| context | sequence containing the reference nucleotide(s) |
| | and the bottom sequence containing the variant |
| | nucleotide(s) |
| Position (Amino | Position within the protein sequence of the amino |
| Acids) | acid encoded by the variant nucleotide. |
| Coding Sequence | The coding region of a gene within the genome |
| Strandedness of the | Determines the coding strand affected by the |
| amino acid affected | change (+ or −) |
| Reference amino | Original amino acid in the reference (3-letter |
| acid | abbreviation and single letter abbreviation) |
| Variant amino | Changed amino acid in the sample (3-letter |
| acid | abbreviation and single letter abbreviation) |
| Synonymous | Yes - amino acids encoded by the reference |
| change | nucleotides and variant nucleotides are identical |
| | No - reference and variant nucleotides encode |
| | different amino acids |
| Amino acid | Original amino acid (single letter abbreviation) |
| change | followed by its position in the CDS and the variant- |
| | encoded amino acid (single letter abbreviation) |
| Protein context | Two 10-amino acid long sequences with the top |
| | sequence containing the reference amino acids and |
| | the bottom sequence containing the variant amino |
| | acids. Empty red box indicates the reference and |
| | variant-encoded amino acids are identical. Green |
| | filled red box indicates the reference and variant- |
| | encoded amino acids differ. |
| Amino acid | Property of the variant-encoded amino acid (when |
| property change | it differs from the original sequence) |
| Change effect | Effect of change to the amino acid sequence |
| | encoded by the variant. |

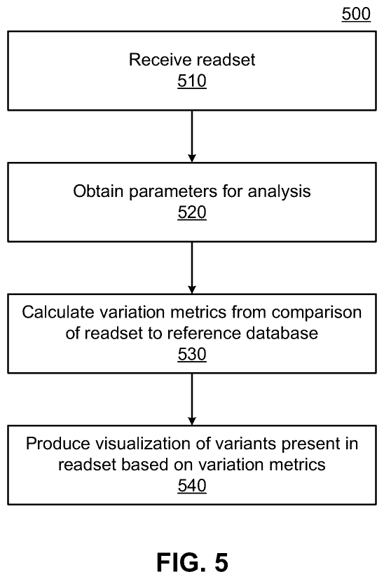

FIG. 5 illustrates an example method 500 that may be performed by the SNV module 260, according to one embodiment. Although the steps of FIG. 5 are described from the perspective of the SNV module 260 performing the method 500, some or all of the steps may be performed by other entities or components. In addition, some embodiments may perform steps in parallel, perform steps in different orders, or perform different steps.

In the embodiment shown in FIG. 5, the method 500 begins with the SNV module 260 receiving 510 one or more readsets. The SNV module 260 also obtains 520 parameters for the analysis. For example, in an embodiment where Bowtie is used for the analysis, the parameters may include one or more of: alignment mode (local or end to end); reporting options (default, a-report all, k-select number); or presets (fast, very-fast, sensitive, or very-sensitive). The parameters may also include parameters for the variant caller used. For example, in an embodiment where LoFreq is used, the parameters may include one or more of: whether to enable or disable indel calls; what P-value cutoff to use; the depth at which to cap coverage; or whether to merge mapping quality in the LoFreq model. It should be appreciated that a wide range of parameters and models may be used.

Once the readsets and parameters have been selected, the SNV module 260 calculates 530 variation metrics by comparing the readsets to the reference database. The variation metrics may include, for each nucleotide position, one or more of the frequency with which the sequences of the reads match the reference genome, an absolute number of reads that do not match the reference genome, a number of reads in the readset that provide coverage at that nucleotide position, a distribution of what substitution, deletion, or insertion is present for reads that do not match the reference genome, or the like. The SNV module 260 produces 540 one or more visualizations of the variations present using the metrics. For example, as described previously, the SNV module 260 may produce a plot of coverage and variation percentage by position and a data table, either or both of which may be made available in a tab of the user interface provided by the interface module 210. It should be appreciated that a wide range of visualizations of variation data are possible, depending on the specific needs of the use case and the preferences of the user.

Referring again to FIG. 2, the datastores are one or more non-transitory computer-readable media that store data used by the modules of the analysis system 120. In one embodiment, one datastore stores readsets 270, another datastore stores the reference data 280, and another datastore stores a run history 290. The run history 290 includes the results of previous analyses performed by the analysis system 120. Thus, a user may review previous results without the analysis system 120 having to reperform the corresponding analysis. In one embodiment, all analyses performed are stored in the run history. Alternatively, in some embodiments, results may be stored on demand and/or results may be deleted from the run history 290 if certain criteria are met. For example, results may be deleted if they are older than a predetermined age (e.g., one year), have not been accessed for a specified time (e.g., six months), or on the request of the responsible user, etc.

### Computing System Architecture

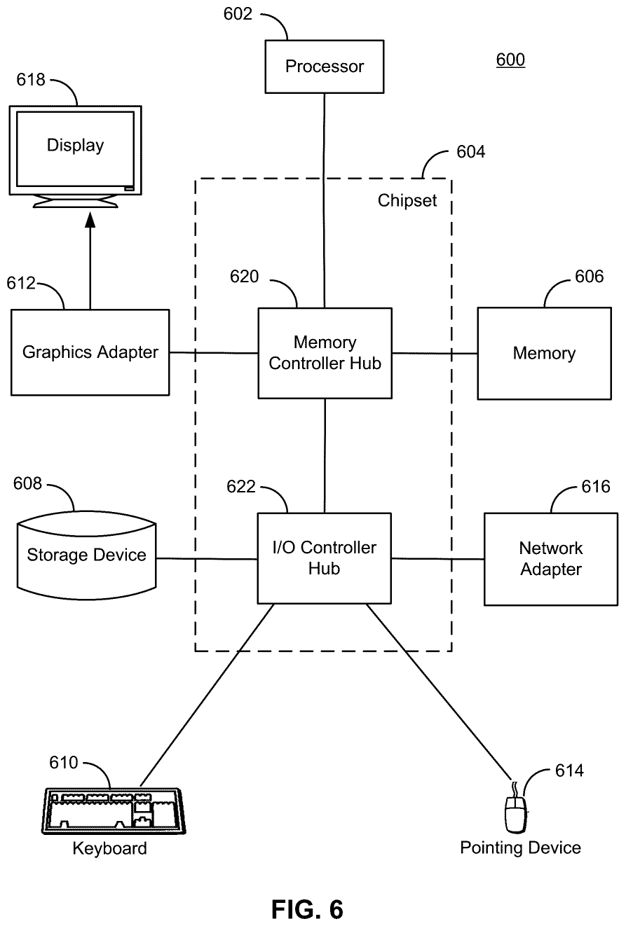

FIG. 6 is a block diagram of an example computer 600 suitable for use as part of a sequencing system 110, an analysis system 120, or a client device 140. The example computer 600 includes at least one processor 602 coupled to a chipset 604. The chipset 604 includes a memory controller hub 620 and an input/output (I/O) controller hub 622. A memory 606 and a graphics adapter 612 are coupled to the memory controller hub 620, and a display 618 is coupled to the graphics adapter 612. A storage device 608, keyboard 610, pointing device 614, and network adapter 616 are coupled to the I/O controller hub 622. Other embodiments of the computer 600 have different architectures.

In the embodiment shown in FIG. 6, the storage device 608 is a non-transitory computer-readable storage medium such as a hard drive, compact disk read-only memory (CD-ROM), DVD, or a solid-state memory device. The memory 606 holds instructions and data used by the processor 602. The pointing device 614 is a mouse, track ball, touchscreen, or other type of pointing device, and may be used in combination with the keyboard 610 (which may be an on-screen keyboard) to input data into the computer system 600. The graphics adapter 612 displays images and other information on the display 618. The network adapter 616 couples the computer system 600 to one or more computer networks, such as network 170.

The types of computers used by the entities of FIGS. 1 and 2 can vary depending upon the embodiment and the processing power required by the entity. For example, the analysis system 120 might include multiple blade servers working together to provide the functionality described. Furthermore, the computers can lack some of the components described above, such as keyboards 610, graphics adapters 612, and displays 618.

### ADDITIONAL CONSIDERATIONS

Some portions of above description describe the embodiments in terms of algorithmic processes or operations. These algorithmic descriptions and representations are commonly used by those skilled in the computing arts to convey the substance of their work effectively to others skilled in the art. These operations, while described functionally, computationally, or logically, are understood to be implemented by computer programs comprising instructions for execution by a processor or equivalent electrical circuits, microcode, or the like. Furthermore, it has also proven convenient at times, to refer to these arrangements of functional operations as modules, without loss of generality.

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate+/−10% unless another meaning is apparent from the context. From example, “approximately ten” should be understood to mean “in a range from nine to eleven.”

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs. Thus, while particular embodiments and applications have been illustrated and described, it is to be understood that the described subject matter is not limited to the precise construction and components disclosed. The scope of protection should be limited only by the following claims.

What is claimed is:

1. A system for processing biomolecule sequencing data, the system comprising:
one or more processors;
an interface module that interacts with the one or more processors to obtain biomolecule sequencing data comprising a plurality of reads; and
one or more tools that interact with the one or more processors to process the biomolecule sequencing data, the one or more tools comprising:
a short read alignment module that classifies the plurality of reads into subsets based on alignment to one or more reference genomes,
wherein the interface module further provides a web-based interface that causes a client device to display results of analysis performed by the one or more tools, the results including data describing the subsets of the plurality of reads.

2. The system of claim 1, wherein, to classify the plurality of reads into subsets, the short read alignment module:
receives a readset including the plurality of reads;
preprocesses the readset;
attempts alignment of the plurality of reads with one or more reference genomes; and
classifies the plurality of reads into the subsets based on results of the attempts to align the plurality of reads.

3. The system of claim 2, wherein the subsets comprise aligned, half-aligned, and unaligned relative to the one or more reference genomes, wherein aligned reads are ones that generate complete matches with the one or more reference genomes, half-aligned reads are ones for which only one part are aligned with the one or more reference genomes, and unaligned reads are any other reads.

4. The system of claim 2, wherein the short read alignment module preprocesses the readset by performing one or more operations that converts the readset into a predetermined format, the one or more operations including at least one of deduplicating read names to provide each sequence read a unique identifier or removing suffixes from read names.

5. The system of claim 2, wherein the short read alignment module provides a plurality of aligners for alignment of the plurality of reads with the one or more reference genomes, and the user interface comprises controls for a user to select one or more of the plurality of aligners for use by the short read alignment module in analysis of the plurality of reads.

6. The system of claim 2, wherein the short read alignment module further:
calculates alignment statistics for one or more reference organisms represented in the one or more reference genomes with which at least some of the sequence reads were aligned;
enriches the alignment statistics using a taxonomy for the one or more reference organisms;
generates one or more coverage plots for the sequence reads relative to the one or more reference organisms; and
builds one or more consensus contigs from the sequence reads for the one or more reference organisms.

7. The system of claim 6, wherein the alignment statistics are enriched by adding, to a file including the alignment statistics, one or more of: the taxonomy, a total number of reads per family, a total number of reads per genus, or a total number of reads per species.

8. The system of claim 1, wherein the one or more tools further comprise a protein alignment module that matches sequences of amino acids in the sequencing data to reference proteins.

9. The system of claim 1, wherein the one or more tools further comprise a de novo assembly module that builds a genome from the sequence data.

10. The system of claim 9, wherein the de novo assembly module builds the genome from the sequence data by performing a series of operations to:
receive the plurality of reads;
produce aligned contigs, relative to the one or more genomes, from the plurality of reads;
generate coverage plots indicating coverage of the one or more reference genomes in the plurality of reads; and
provide a taxonomy table generated from the aligned contigs and the coverage plots, the taxonomy table identifying organisms identified in the plurality of reads.

11. The system of claim 10, wherein the taxonomy table includes clickable links to the underlying sequencing data for each organism.

12. The system of claim 1, wherein the one or more tools further comprise a single nucleotide variation module that identifies variations in the plurality of reads relative to the one or more reference genomes and generates a visualization representing the variations.

13. The system of claim 12, wherein the visualization is a variations plot that indicates, for each position in a genome of the one or more genomes, metrics describing an amount of coverage for that position in the plurality of reads and a degree of variation between the reads in the plurality of reads and the genome.

14. The system of claim 12, wherein the visualization is a table that includes one or more of: a nucleotide position, a reference allele, a variant allele, a variant type, a frequency, nucleotide context, an amino acid position, a coding sequence, a strandedness of an affected amino acid, a reference amino acid, a variant amino acid, an indication of whether the change is synonymous, an amino acid change, a protein context, an amino acid property change, or a change effect.

15. The system of claim 12, wherein the single nucleotide variation module generates the visualization by a series of processes that:
receive the plurality of reads;
obtain parameters for analysis;
calculate variation metrics with a comparison of the plurality of reads to the one or more reference genomes; and
produce the visualization using the variation metrics.

16. The system of claim 1, further comprising a sequencing system connected to the interface module via a network, wherein the sequencing system generates the plurality of reads from one or more biological samples and transfers the plurality of reads to the interface module via the network.

17. The system of claim 16, wherein the sequencing system comprises a next generation sequencer.

18. The system of claim 1, further comprising a curation module that curates the one or more reference genomes.

19. The system of claim 18, wherein the curation module tags a portion of a reference genome of the one or more reference genomes as corresponding to contamination, noise, or a low-complexity region, and the portion is ignored or given a lower weight in analysis by the short read alignment module.

20. The system of claim 1, wherein the web-based interface comprises a plurality of tabs, each tab providing results of analysis of a different tool of the one or more tools.
